(19) United States
(12) Patent Application Publication
(10) Pub. No.: US 2024/0016384 A1
(43) Pub. Date: Jan. 18, 2024

(54) MAGNETIC RESONANCE IMAGING (MRI) RECEIVE COIL COMPATIBLE WITH MRI GUIDED HIGH INTENSITY FOCUSED ULTRASOUND (HIFU) THERAPY

(71) Applicant: The Regents of the University of California, Oakland, CA (US)

(72) Inventors: Shimon Michael Lustig, Moraga, CA (US); Ana Claudia Arias, Lafayette, CA (US); Joseph R. Corea, Berkeley, CA (US); Anit M. Flynn, Oakland, CA (US)

(21) Appl. No.: 18/358,754

(22) Filed: Jul. 25, 2023

Related U.S. Application Data
(62) Division of application No. 16/656,380, filed on Oct. 17, 2019, now abandoned.
Application No. 16/656,380 is a continuation of application No. PCT/US2018/028541, filed on Apr. 20, 2018.
(60) Provisional application No. 62/487,900, filed on Apr. 20, 2017.

(51) Int. Cl. A61B 5/00; A61B 5/055; A61N 7/02; G01R 33/34; G01R 33/48

(52) U.S. Cl. CPC A61B 5/0036; A61B 5/055; A61N 7/02; G01R 33/34007; G01R 33/34084; G01R 33/4814

(57) ABSTRACT

Magnetic Resonance Imaging (MRI) receiver coil devices, including a MRI receiver coil or MRI receiver coil arrays, for use in a MRI guided High Intensity Focused Ultrasound system, and methods for manufacturing the same. A MRI receive coil device includes a flexible substrate having a first surface and a second surface opposite the first surface, and a pattern of conductive material formed on one or both of the first and second surfaces, the pattern including at least one receive coil and at least one capacitor, wherein the flexible substrate comprises a dielectric plastic material. In certain aspects, at least one layer of hydrophobic material covers the at least one receive coil and the at least one capacitor.

### CROSS REFERENCES TO RELATED APPLICATIONS

This patent application is a Divisional of U.S. application Ser. No. 16/656,380 by Lustig et al., entitled “MAGNETIC RESONANCE IMAGING (MRI) RECEIVE COIL COMPATIBLE WITH MRI GUIDED HIGH INTENSITY FOCUSED ULTRASOUND (HIFU) THERAPY,” filed Oct. 17, 2019, which is a continuation of PCT Application No. PCT/US2018/028541 by Lustig et al., entitled “MAGNETIC RESONANCE IMAGING (MRI) RECEIVE COIL COMPATIBLE WITH MRI GUIDED HIGH INTENSITY FOCUSED ULTRASOUND (HIFU) THERAPY,” filed Apr. 20, 2018, which claims priority to U.S. Provisional Patent Application No. 62/487,900 by Lustig et al., entitled “MAGNETIC RESONANCE IMAGING (MM) RECEIVE COIL COMPATIBLE WITH MRI GUIDED HIGH INTENSITY FOCUSED ULTRASOUND (HIFU) THERAPY,” filed Apr. 20, 2017, each of which is incorporated herein by reference in its entirety.

### FEDERALLY SPONSORED RESEARCH OR DEVELOPMENT

This invention was made with Government support under Grant Number R21EB015628, awarded by the National Institute of Health. The Government has certain rights in this invention.

### BACKGROUND

The present disclosure generally provides Magnetic Resonance Imaging (MRI) receiver coil devices, including a MRI receiver coil or MRI receiver coil arrays, and methods for manufacturing the same, and more particularly MRI receive coil devices useful in MRI guided High Intensity Focused Ultrasound (HIFU) therapy techniques.

In MRI, very small signals are created via excitation of hydrogen protons in the bore of an MRI machine. These signals are picked up on receiver coils adjacent to the patient inside the machine and processed to yield an image. The higher the signal-to-noise (SNR) the receiver coils can produce, the faster the scan time can be and the higher the quality of images that can be produced. MRI receiver coil arrays provide a better signal-to-noise-ratio and field of view over standard single coil receivers. However, this gain is lost when the surface coil array is at an improper distance from the patient.

MRI guided High Intensity Focused Ultrasound (HIFU) is a therapy technique used to ablate tissue or activate heat sensitive medication inside a patient's body with acoustic energy while being tracked (i.e., guided) with images from an MRI scanner. This technique successfully treats uterine fibroids, drastically reduces the pain from bone cancer metastases, and dramatically reduces essential tremor. This quickly expanding field has shown promise for the treatment of other conditions including brain conditions, where classical imaging techniques struggle to guide without using an invasive borehole in the patient's head. Currently, a major limiting factor of MRI guided HIFU is the precision and speed of the imaging hardware used to track treatment areas. Specifically, the state-of-the-art receive coils in a MRI scanner are incompatible with the ultrasonic transducer, so a less effective body coil with lower image quality must be used.

A more effective solution is a surface coil, which has extremely high signal to noise ratio and enables accurate temperature monitoring at high resolution. A surface coil is only sensitive to tissue close to the coil, so it must be placed between the transducer and the patient to be effective. However, to treat an entire target, the transducer is moved in the water bath, which would pass acoustic energy directly through different parts of the surface coil. Ultrasonic energy easily scatters and attenuates in the thick fiberglass reinforced boards, solder, and porcelain capacitors commonly used in coil construction (FIG. 1). Therefore, current surface coils are not suitable for such use and only body coils are used.

There is therefore a need for MRI receiver coil devices that provide increased SNR, and which are compatible with HIFU techniques and instruments. There is also a need for cost-effective fabrication processes for forming such receiver coil devices.

### SUMMARY

The present embodiments provide surface coil arrays that are transparent to acoustic energy and which drastically increase image quality and temperature estimation. Advantageously, these device embodiments can be used in MRI guided HIFU of the head or body, specifically for the treatment of brain conditions (including essential tremor), cancer, and uterine fibroids. In certain aspects, the device is completely waterproof and able to be submerged for extended periods of time. Imaging aquatic animals may be possible without removing them from water.

According to an embodiment, a flexible magnetic resonance imaging (MRI) receive coil device for use in a MRI guided High Intensity Focused Ultrasound system is provided. The MRI receive coil device typically includes a flexible substrate having a first surface and a second surface opposite the first surface, and a pattern of conductive material formed on one or both of the first and second surfaces, the pattern including the at least one receive coil and the at least one capacitor, wherein the flexible substrate comprises a dielectric plastic material selected from the group consisting of a polyimide (PI) film, a polyethylene (PE) film, a polyethylene terephthalate (PET) film, a polyethylene naphthalate (PEN) film, a polyetherimide (PEI) film, a polyphenylene sulfide (PPS) film, a polytetrafluoroethylene (PTFE) film, and a poly ether ketone (PEEK) film. In certain aspects, the MRI receive coil device further includes at least one layer of hydrophobic material covering the at least one receive coil and the at least one capacitor. In certain aspects, the at least one receive coil and the at least one capacitor are substantially transparent to ultrasound frequencies. In certain aspects, the MRI receive coil device further includes at least one layer of material covering the at least one receive coil and the at least one capacitor, wherein the at least one layer of material has an acoustic impedance between an acoustic impedance of water and an acoustic impedance of the conductive material. In certain aspects, a thickness of the MRI receive coil device is less than about 0.1 mm (e.g., between about 0.01 mm and 0.1 mm).

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE SEVERAL VIEWS OF THE DRAWING(S)

The detailed description is described with reference to the accompanying figures. The use of the same reference numbers in different instances in the description and the figures may indicate similar or identical items.

FIG. 1 illustrates a patient in a HIFU capable scanner with cross-section; the ultrasonic transducer is in a water bath below patient's body.

FIG. 2A shows a setup and acoustic power distribution from transducer as seen by a hydrophone over a 20×20 mm² area; printed coil structures according to embodiments do not attenuate or distort the signal significantly whereas conventional materials do distort the signal significantly.

FIG. 2B shows the maximum signal intensity of ultrasonic signals transmitted through a printed coils according to embodiments and conventional coil materials; current coil materials significantly attenuate ultrasonic energy.

FIG. 3A shows a picture of HIFU compatible printed (flexible) coil according to an embodiment.

FIG. 3B shows a sagittal scan of an InSightec heating phantom using the printed coil.

FIG. 3C shows SNR vs. depth into the phantom for printed and body coils showing superior printed surface coil performance; the body coil is the current standard imaging technique for MRI guided ultrasound therapy.

FIG. 4A shows a flexible printed coil array according to an embodiment.

FIG. 4B shows a cross-section summary of a printing process for fabricating the flexible printed coil array according to an embodiment.

FIG. 5 shows an example of a flexible surface array according to an embodiment, highlighting how the conductive traces sandwich the plastic substrate to form very thin capacitors.

FIG. 6A shows the change in the Q value and FIG. 6B shows the change in the resonant frequency that the coils experienced before and after submersion in water for 24 hours.

FIG. 7A shows a transducer passing acoustic power through test films to a hydrophone that records the acoustic intensity to characterize the test films.

FIG. 7B shows the relative acoustic power measured from several samples of PEEK at 650 kHz and 1 MHz—frequencies common to head and body MRI guided ultrasound therapy, respectively.

FIG. 7C shows the relative acoustic power measured through several samples of silver ink on PEEK film at 650 kHz and 1 MHz.

FIG. 7D shows the percentage of power transmitted through a PTFE/PEEK/PTFE test film over a span of frequencies.

FIG. 7E shows the 2D acoustic power transmission profiles for a printed capacitor of the present disclosure in addition to the traditionally used coil circuit and encapsulation materials.

FIG. 8A illustrates the positioning of a printed array, according to an embodiment, wrapped around a gel phantom and submerged inside a head transducer to characterize the SNR.

FIG. 8B and FIG. 8C show the SNR across the center of the phantom, which shows that the array of the present embodiment presents 5 times the SNR at the surface of the phantom when compared to the currently used body coil.

FIG. 8D shows a comparison between the abdominal images from the body coil and the transparent arrays, which shows that it is possible to obtain images with more detailed liver and stomach regions when using the printed array of the present embodiment.

FIG. 8E shows axial and coronal slices of the maximum heating point for ultrasonic heating experiments.

FIGS. 9A-F show heating and imaging experiment and results using a printed coil array according to an embodiment.

FIG. 9A is an annotated scan that illustrates how the coil is placed in-between the transducer and the phantom during these experiments.

FIG. 9B shows examples of the temperature maps taken with the body coil without and with the 4-channel array present.

FIG. 9C shows the thermometry maps inside the gel phantoms with and without the coil present.

FIG. 9D illustrates the positioning of the 4-channel array on the skull phantom while it was heated inside a head transducer.

FIG. 9E shows the temperature map overlaid on the anatomy scan of the bovine brain; the temperature map in FIG. 9E is similar to the heating profile shown in FIG. 8E, indicating there is not significant distortion or attenuation due to the array of the present embodiment.

FIG. 9F shows a high-resolution scan of the brain phantom taken inside the transducer.

### DETAILED DESCRIPTION

The following detailed description is exemplary in nature and is not intended to limit the invention or the application and uses of the invention. Furthermore, there is no intention to be bound by any expressed or implied theory presented in the following detailed description or the appended drawings.

Turning to the drawings, and as described in greater detail herein, embodiments of the disclosure provide surface coil arrays that are transparent to acoustic energy and which drastically increase image quality and temperature estimation.

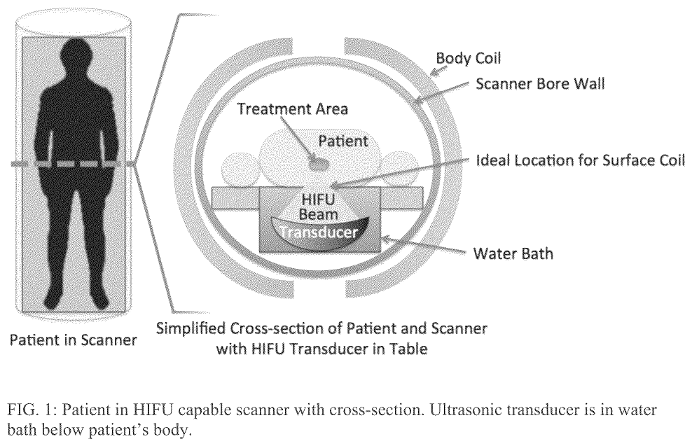

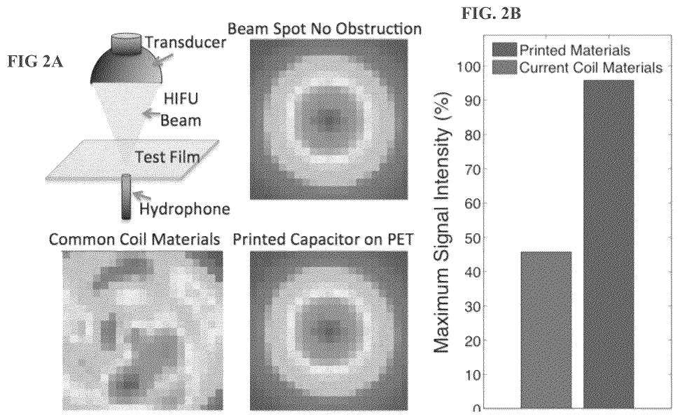

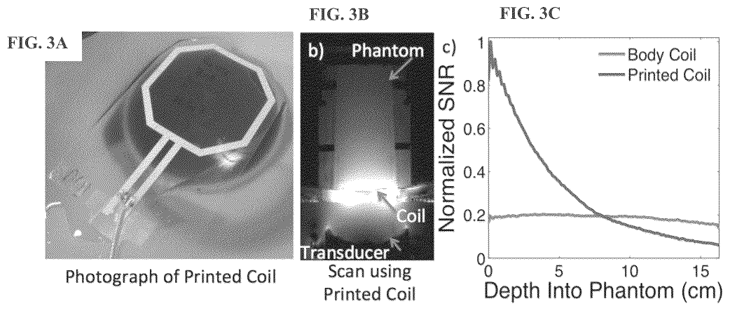

In certain embodiments, screen printing techniques are used to make a coil array for an MRI scanner that is extremely thin (e.g., less than 0.1 mm) and renders the coil array nearly invisible to MRI guided High Intensity Focused Ultrasound (HIFU), a therapy used to ablate tumors inside the human body. This allows for the coil to be inserted directly in the beam path of the ultrasonic energy, drastically increasing the quality of images used to guide the treatment. (see, FIG. 1, FIG. 2 and FIG. 3). Such a HIFU compatible array enables array based imaging acceleration techniques (such as parallel imaging) to be used in ultrasound therapy. In certain embodiments, HIFU compatible receive coils arrays for MRI scanners are fabricated using additive solution processing techniques to print (form) conductors, insulators, capacitors, inductors, transmission lines and other discrete devices needed for their proper function. Coil materials and packaging are made to tolerate being submerged in water, essential to functioning during the therapy. In some embodiments, for example, the materials used are optimized for water submersion over an expanded period of time and/or the device may be coated with a hydrophobic or waterproofing material. Coils can be tuned for human scanning systems, specifically 1.5 T, 3 T, but can easily be adapted for 7 T.

In certain embodiments, MRI coils are fabricated on a flexible substrate or thin film. Examples of flexible substrate materials include thin films of PET (Polyethylene terephthalate), Kapton (polyimide or PI), PEN (Polyethylene napthalate) sheet, or PEEK (Polyether ether ketone). Prior to printing, the substrate may be preheated to the temperature experienced during annealing to relieve any stress and prevent distortion in future processing steps. The substrate is then allowed to cool to room temperature before proceeding onto the printing process.

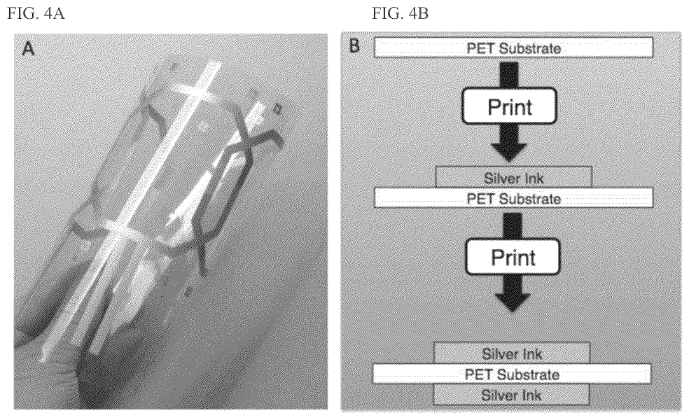

Printing the conductive layers is accomplished in certain embodiments by printing, e.g., screen-printing a conductive ink, such as a silver microflake ink, onto the substrate followed by annealing, e.g., 125° C. anneal for 15 min. Thereafter, the substrate is overturned and the overturned substrate is loaded back into the screen printer to receive the same patterning on the back. A schematic of the processing steps is shown in FIG. 4B. Coils then received a waterproof coating to prevent degradation in the water environment. U.S. Provisional Application Ser. No. 62/469,253, filed on Mar. 19, 2017, and PCT Application PCT/US2018/021820, filed Mar. 9, 2018, which are both hereby incorporated by reference, provide additional details regarding MRI receiver coil fabrication processes and materials.

Traditional surface coils are not compatible with MRI guided ultrasound therapy, but the coils of the present disclosure advantageously fill that performance gap and would aid doctors in observing the treatment area with higher resolution than ever before (including with higher resolution in time), potentially reducing complications and surgery time.

Drastically improving the utility of MRI guided ultrasound therapy would greatly increase the market for this therapy, bringing life changing treatment to more patients.

These MRI guided ultrasound therapy compatible coils drastically increase the resolution of the images doctors use to monitor the treatment at a higher monitoring rate. These coils interface in the same way other traditional surface coils interface with the scanner, requiring little to no retrofitting of existing equipment for their use. Ultrasonic image guiding is a potential alternative to an MRI guided image (and would not require a receive array), however this tracking technique does not work well though the skull, so MRI guided ultrasound therapy is still a better alternative for the head.

The present embodiments provide surface coil arrays that are transparent to acoustic energy and which drastically increase image quality and temperature estimation. One way to fabricate an acoustically transparent coil is to use very thin polymer-based materials and solution processed conductors. These materials can be selected to have acoustic properties close to that of water reducing the amount of interaction with the acoustic energy. Such coils may be fabricated using screen-printed conductive inks on thin plastic substrates. A surface coil is a resonant loop of wire tuned to resonate at the Larmor frequency of the scanner using in-series capacitors. To fabricate these coils, solution processed conductors are selectively deposited in a loop on a flexible plastic substrate with tuning capacitors. Reference is made to U.S. Provisional Application Ser. No. 62/469,253, filed on Mar. 19, 2017, which is incorporated by reference in its entirety, for additional and supplemental information regarding MRI receiver coils, fabrication processes and materials.

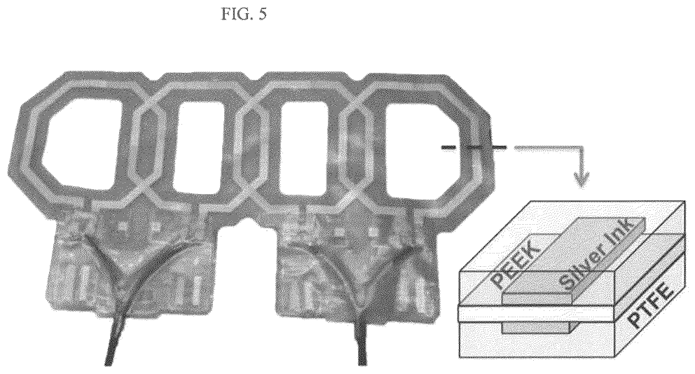

FIG. 5 shows an example of a flexible surface array according to an embodiment, highlighting how the conductive traces sandwich the plastic substrate to form very thin capacitors. The capacitance depends on the amount of overlap, substrate material, and substrate thickness. The printing and ink drying processes use temperatures between 80-140° C., allowing for a wide variety of common plastics to be used for coil fabrication.

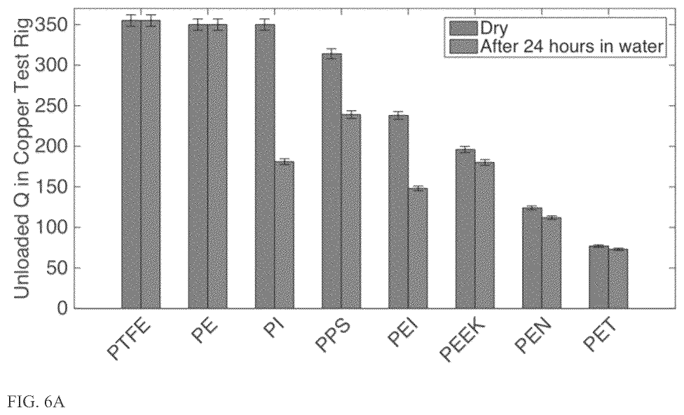

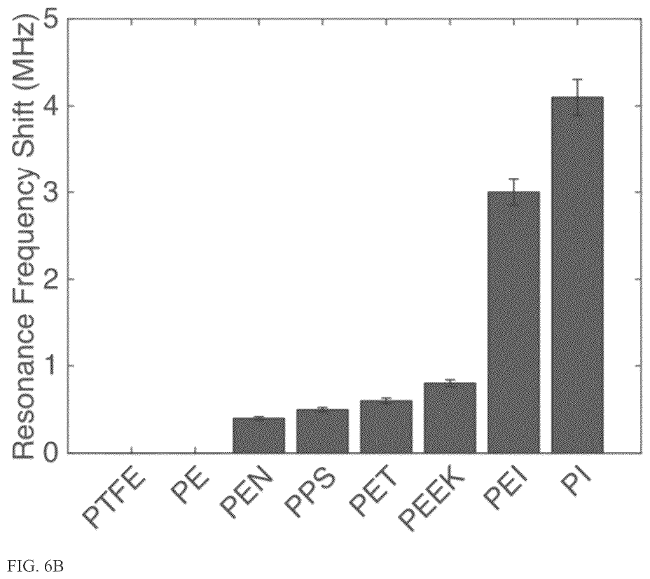

In certain embodiments, polytetrafluoroethylene (PTFE), polyethylene (PE), polyimide (PI), polyphenylene sulfide (PPS), polyetherimide (PEI), polyether ether ketone (PEEK), polyethylene naphthalate (PEN) and polyethylene terephthalate (PET) are used as substrate materials. FIG. 6A shows the change in the Q value and FIG. 6B shows the change in the resonant frequency that the coils experienced before and after submersion in water for 24 hours. Any change in Q before and after submersion is more important than the maximum Q value for any particular substrate. Material properties that vary with exposure to water make tuning the coil challenging as any absorbed water changes the coil tuning which significantly degrades image SNR. For example, PI, PPS, and PEI show higher Q than PEEK, but after submersion in water the resonant frequency and Q significantly change. The shift in the coil tuning is due to the large difference in dielectric constants between plastics (εr≈2-4) and water (εr=80 at 20° C.), therefore even a small amount of absorbed water has a large impact on the resonant frequency. Other substrates such as PE and PTFE show high Q values with very small shift, but are not as desirable for the printing process due to poor adhesion of the conductive ink and are easily deformed by mechanical stress. According to an embodiment, a PEEK substrate is a desirable material to fabricate MRI guided ultrasound therapy coils due to its high Q, low water absorption, and conductive ink compatibility.

In one embodiment, DuPont 5064 H silver ink is used for the conductive portions of the coil. Other conductive inks or conductive materials may be used for the conductive portions of the coil. After 24 hours of water submersion, the samples made of the DuPont 5064 H silver ink did not experience any significant change in resistivity; showing resistivity of 16±2 μohm-cm before and after. Furthermore, the surface roughness of the ink did not change, maintaining a root mean squared (RMS) surface roughness of 1.3±0.2 μm both times.

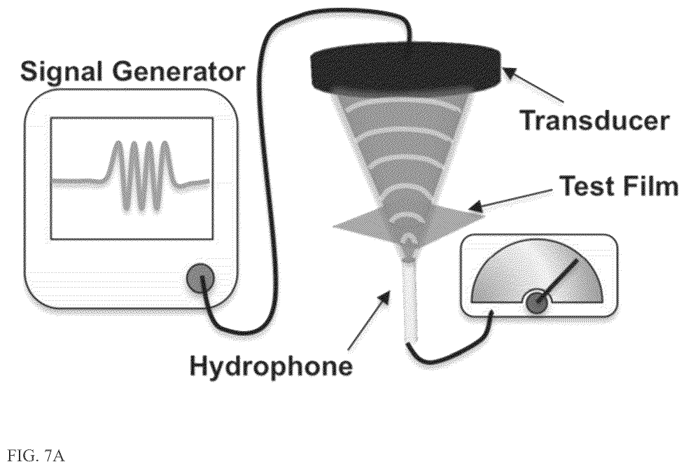

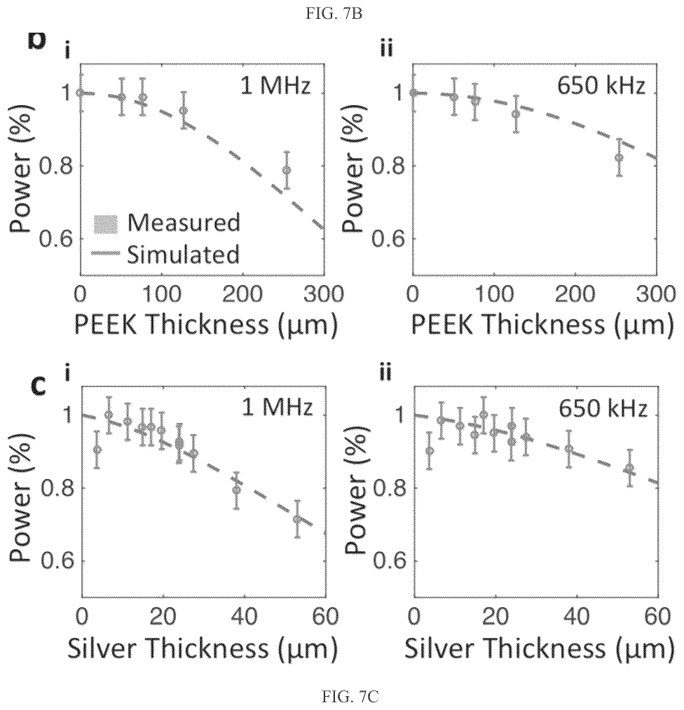

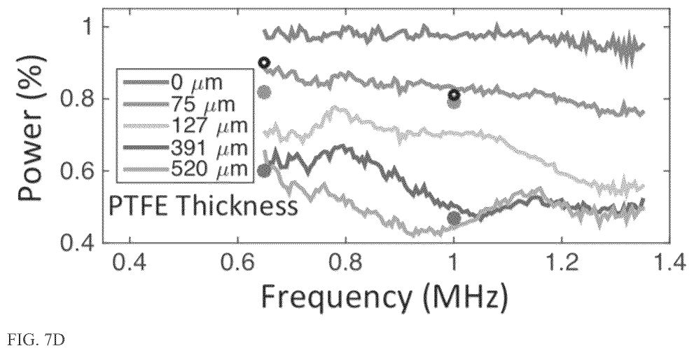

The coil materials used should also transmit a high percentage of incident acoustic energy without distortion. Local surface burns, damage to the transducer, and low focal heating may occur if the coils reflect or attenuate a significant amount of the acoustic energy. To characterize the films, a transducer passes acoustic power through test films to a hydrophone that records the acoustic intensity, as illustrated in FIG. 7A.

The acoustic absorption of PEEK is characterized in the thickness range of 50 μm to 254 μm to determine the optimal thickness. All film thicknesses are within 10% of the reported values. FIG. 7B shows the relative acoustic power measured from several samples of PEEK at 650 kHz and 1 MHz—frequencies common to head and body MRI guided ultrasound therapy, respectively. It can be seen that the thinnest films of PEEK provide the least amount of attenuation; however, thinner films are more difficult to process as they are more susceptible to mechanical damage.

As a result, a PEEK film thickness of 76 μm was selected to maintain acoustic transparency, handling robustness, and ease of processing. Other thicknesses of PEEK, e.g., ranging from 10 μm to 300 μm or greater, may be used, as may a variety of thicknesses of other materials as will be appreciated by one skilled in the art.

The acoustic properties of solution-processed materials are not commonly available. To determine the acoustic impedance of the conductive silver ink acoustic power was transmitted though several thicknesses (3-56 μm) of the silver film deposited on the 76 μm of PEEK film. FIG. 7C shows the relative acoustic power measured through several samples of silver ink on PEEK film at 650 kHz and 1 MHz. Also shown in FIG. 7B and FIG. 7C, are the results from simulations using an acoustic model. The measured values of transmitted acoustic power are in agreement with the predicted transmitted power, suggesting that the printed silver films are attenuating the acoustic energy mainly by transmission and reflection interactions rather than by diffuse scattering or bulk attenuation. By fitting the data to the acoustic model it was found that the DuPont 5064 H silver ink has an acoustic impedance of 15.6±3.8 MRayls. This value is closer to that of water at 1.5 MRayls, when compared to commonly used copper at 44.6 MRayls or bulk silver at 38.0 MRayls. This decreased acoustic impedance can be attributed to the composition of the ink, which is composed of a suspension of silver micro-flakes into polymer-based binders that remain in the film after the thermal curing process. The silver microflakes in the ink have an acoustic impedance similar to bulk silver while the polymer binders have a lower acoustic impedance, similar to most plastics. Combining the two gives acoustic properties in between the two constituent materials, like those shown in the measurement. The decreased acoustic impedance allows reduced reflections at any water, tissue, or plastic interface compared to commonly used conductors. If higher acoustic transparency were desired, the ink could be reformulated to increase the load of low acoustic impedance materials in the solution. There would be a trade-off between conductivity and acoustic transparency. Overall the acoustic properties of the commercially available silver ink make it well suited for use in the acoustically transparent coils.

To protect the patient from any DC bias that might exist on the coil, an electrically isolating film is deposited over the conductive traces in an embodiment. This film should be acoustically transparent in addition to providing high electrical breakdown strength. A PTFE film was selected as an appropriate material for further characterization and optimization. Test films with 75, 127, 391, and 520 μm in thickness of PTFE were measured for transmission across a span of common MRI guided ultrasound therapy frequencies.

FIG. 7D shows the percentage of power transmitted through the PTFE/PEEK/PTFE test film over a span of frequencies. The highest transmission across all frequencies is given by 76 μm of PTFE film on both sides of the 76 μm PEEK substrate. As a result, this stack is used as a desirable coil construction, although one skilled in the art will recognize that other stack dimensions and materials may be used.

The optimized material stack of a 76 μm thick PEEK substrate encapsulated in 76 μm of PTFE with 15 μm of the printed conductor is further characterized by comparing it to the traditional materials used in coil construction. FIG. 7E shows the 2D acoustic power transmission profiles for a printed capacitor of the present disclosure in addition to the traditionally used coil circuit and encapsulation materials. From these 2D acoustic pressure maps, no significant distortion or scattering in the focal spot for the printed capacitor was noticeable. The printed capacitor transmitted 80.5% of the acoustic power at 1 MHz and 89.5% at 650 kHz, in agreement with previous testing. These transmissions are much higher compared with the 51.4% and 62.5% obtained with the 2 mm thick acrylic. The beam shape is also preserved for both the acrylic and printed capacitors, but it is significantly scattered for the traditionally used porcelain capacitor on copper clad fiberglass reinforced circuit board.

To provide a comparison to a non-printed approach, two commonly available thin copper clad substrates were also evaluated using a hydrophone setup. Commercially available 9 μm copper on top of 50 μm polyimide (Pyralux AP 7156E) and 35 μm copper on top of 50 μm polyimide (Pyralux AP 9121 R) were both encapsulated in 76 μm of PTFE and characterized for comparison to the printed coil. The transmitted acoustic power for these films is shown in FIG. 7D and indicates that while the thinner copper passes 95% of the power compared to the printed coil, the printed coil outperforms the copper coil at both 650 kHz and 1 MHz. In addition to exhibiting poorer acoustic transmission, the Pyralux substrates are made of materials that are sensitive to water. The copper conductors easily corrode and break down if left in water for extended periods of time. The polyimide substrate materials readily absorb water changing the electrical tuning of any coil made from it. For example, when the Pyralux substrate is exposed to water for 24 hours and measured in the Q-testing rig as the other substrates were, the Pyralux absorbed enough water to drop the Q from 356 to 232 and shift the resonant frequency 2.5 Mhz.

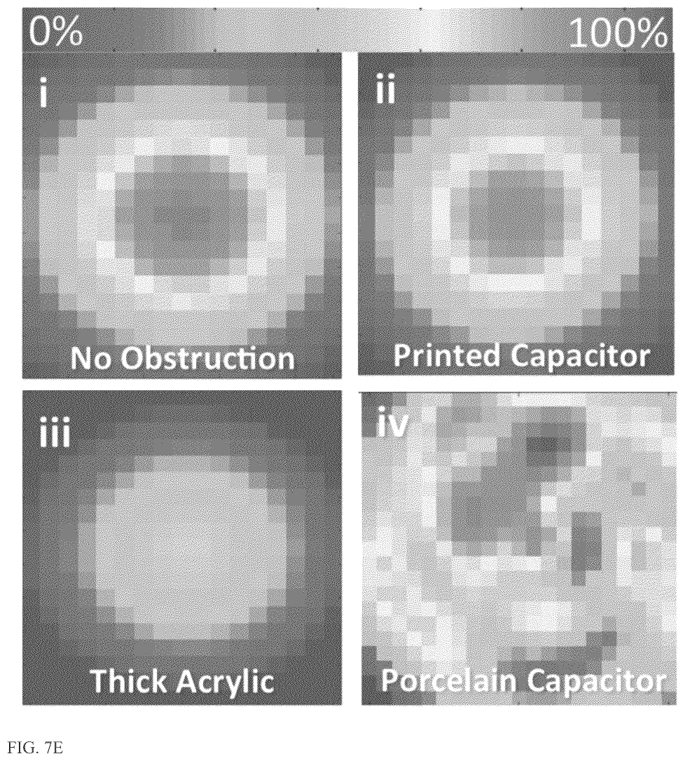

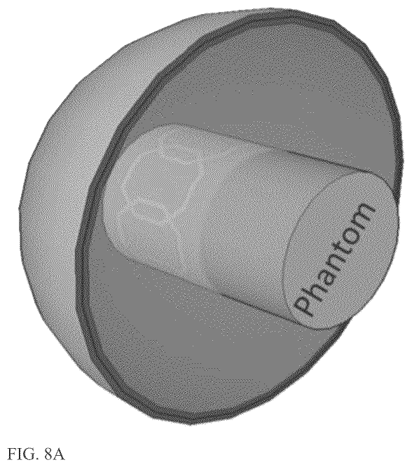

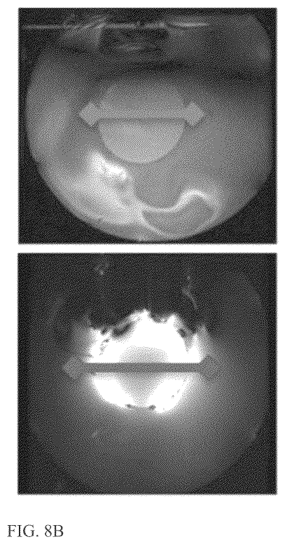

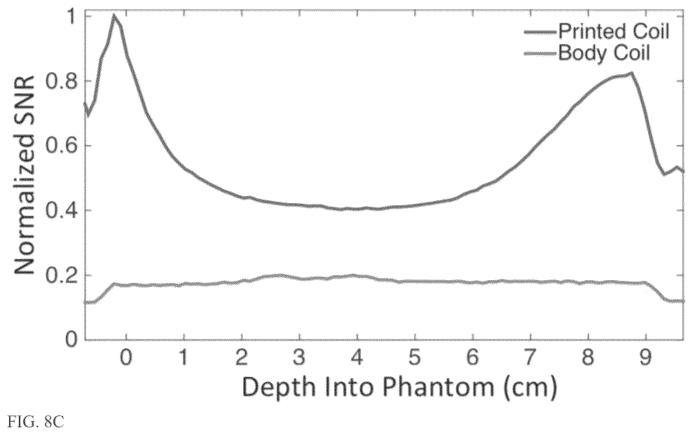

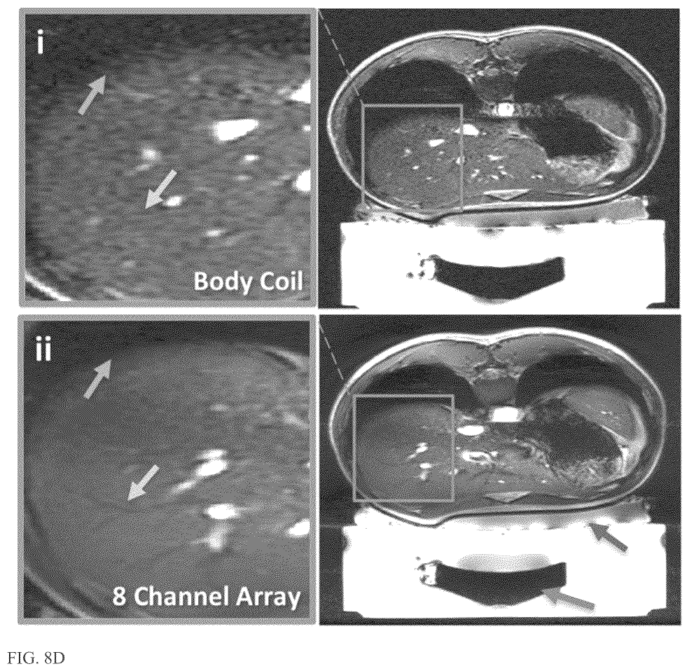

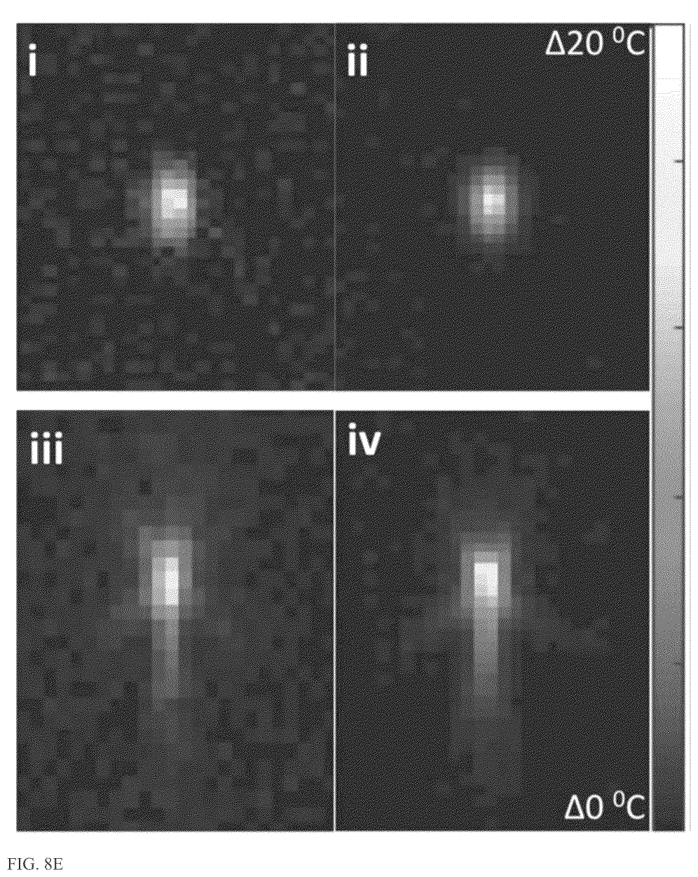

To show that the coils of the present embodiments provide higher SNR than what is currently available in clinical therapy to better guide the procedure, a 4-channel array was fabricated using the optimized material stack of PEEK, PTFE, and silver ink. The SNR of the array is compared to that of the currently used body coil of a 3 T scanner on a gel phantom inside the head transducer. FIG. 8A illustrates the positioning of the printed array wrapped around the gel phantom and submerged inside the head transducer to characterize the SNR. The SNR across the center of the phantom—highlighted in FIG. 8B and FIG. 8C—shows that the array presents 5 times the SNR at the surface of the phantom when compared to the currently used body coil. The asymmetry seen in the coil sensitivity pattern is due to the coil size and the placement on the phantom. At the center of the phantom, where a MRI guided ultrasound therapy procedure is most likely to occur, the array displayed twice the SNR when compared to the body coil. The array also shows more localized sensitivity to the surrounding water and transducer than the body coil, offering additional opportunities to decrease the field of view and shorten the scan time.

To show the clinical SNR gains that a printed coil array according to the present embodiments can provide, breath-hold abdominal images were acquired with an 8-channel coil array wrapped around the abdomen of a volunteer. The comparison between the abdominal images from the body coil and the transparent arrays in FIG. 8D shows that it is possible to obtain images with more detailed liver and stomach regions when using the printed array. Similar to the phantom testing results, the 8-channel array showed the highest SNR at the surface of the volunteer and presents double the SNR in the center of the body. The increased detail would be valuable during treatments and planning surgeries. In addition to the observed SNR benefit, the multichannel array is also able to perform parallel imaging acceleration from the additional channels enabling faster image acquisition.

The array and body coil are used to track ultrasonic heating inside a gel phantom. FIG. 8E shows axial and coronal slices of the maximum heating point for each of these experiments. The heating occurs in the center of the phantom where the 8-channel printed array has slightly more than double the SNR of the body coil. In regions of the phantom that did not see any heating, the standard deviation of temperature estimated was ±0.84° C. from images obtained with the body coil and ±0.19° C. in images from the array. As a result, in both the coronal and axial slices of the heating profile, the coil array provides clearer heating profiles. This is more evident in the coronal profile where the printed array easily shows the side lobes of the heating from the focal point, while the body coil only provides a faint outline of the total profile.

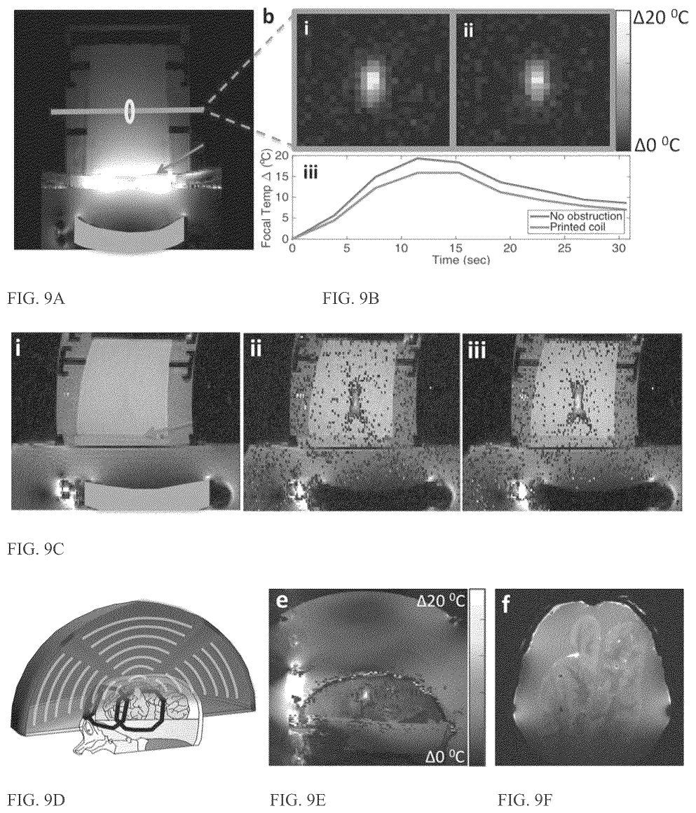

As shown in FIG. 9, the acoustic attenuation of the coil is measured on the scanner by heating an area inside a homogeneous gel phantom to produce approximately 20° C. of temperature rise. For clarity, the annotated scan in FIG. 9A illustrates how the coil is placed in-between the transducer and the phantom during these experiments. The temperature increase is tracked with the body coil of a 3 T scanner with and without the array to maintain the measurement consistency. FIG. 9B shows examples of the temperature maps taken with the body coil without and with the 4-channel array present. When the 4-channel array is placed between the transducer and the phantom, 83±3% of the temperature rise is measured without any noticeable beam distortion. This value matches those seen in the water bath testing along with the acoustic modeling. This 17% attenuation is considerably smaller than the attenuation due to the skull, which is approximately 70%. This attenuation would be much smaller on the 650 kHz head system as suggested by the water bath testing, however the low image SNR from the body coil did not allow precise temperature measurement for this comparison. The transmission of the coil array could be improved if the centers of the coils are removed, but the testing accurately captures the worst case attenuation.

In order to verify that the coils are not absorbing any significant amount of energy that could pose a risk to any nearby tissue, an additional 1.5 cm thick agar gel disk was placed underneath the coil completely surrounding it in material that MR thermometry could be used to measure temperature increase. Next, 54 W of acoustic power was transmitted though the gel stack for 10 seconds with and without the coil present to see if there is any measureable increase temperature near the coil. FIG. 9C shows the thermometry maps inside the gel phantoms with and without the coil present. There is no measurable increase in temperature at or near the coil suggesting that it did not absorb any significant amount of power during the sonication. Afterwards, a second sonication was performed at much lower power to record the amount of reflection seen at the transducer. The amount of reflected signal seen at the transducer was 13% higher with the coil present. This measurement is not directly relatable to how much power is reflected by the coil since not all the reflected energy was captured by the transducer and the signal-to-pressure conversion factor is not well characterized for this analysis, but the increase suggests that the power lost is reflected by the coil water interface rather than absorbed by coil materials.

To demonstrate the proof-of-concept of all system elements together, a 4-channel array was used to track the heating of brain tissue inside the head transducer. A 3D printed ABS plastic skull that mimics bone and containing an ex-vivo bovine brain suspended in a gel was used as a skull phantom. FIG. 9D illustrates the positioning of the 4-channel array on the skull phantom while it was heated inside a head transducer. The temperature map obtained is overlaid on the anatomy scan of the bovine brain in FIG. 9E. The temperature map in FIG. 9E is similar to the heating profile shown in FIG. 8E, indicating there is not significant distortion or attenuation due to the array. Similar to the phantom scans, SNR in the heating region is twice as high as that given by the body coil. Additionally, a high-resolution scan of the brain phantom was taken inside the transducer, shown in FIG. 9F. This scan shows that the highest SNR is at the front of the brain near the coil and slowly drops off towards the back of the head where there is no array. Overall the array shows up to 5 times the SNR at the surface of the body near the coil than the currently used body coil while tracking the heating point inside the skull without significantly attenuating or visibly distorting the acoustic power. For procedures done in the center of the body, the array presented here shows SNR twice as high as the body coil.

The presently disclosed array embodiments advantageously outperform the currently used body coil while tracking the heating point inside the skull without significantly attenuating or visibly distorting the acoustic power.

### Specific Coil Array Fabrication Example

Octagonal coils 8.75 cm in diameter are screen printed onto a plastic substrates using a conductive silver ink (e.g., Dupont 5064 H) patterned through a 165 count stainless steel mesh (e.g., Meshtec). Individual array coils are tuned (e.g., tuned to 127.73 MHz) by changing the area of the in-series capacitors. Coils are then laminated (e.g., in a PTFE film (Professional Plastics)) for water protection, abrasion resistance, and volunteer safety. Coils are connected to a non-printed interface board that contains an inductor and diode to block the coil during the high power RF transmit. A half wavelength long piece of RG-316 non-magnetic cable connects to a box containing preamplifiers (MR Solutions) which then connects to the scanner and/or other processing circuitry or computer.

Reference is also made to U.S. patent application Ser. No. 14/166,679 (US Publication No. 2014/0210466 A1), and U.S. Provisional Application Ser. No. 62/469,253, filed on Mar. 9, 2017, which are each incorporated by reference in its entirety, for additional and supplemental information regarding MRI receiver coils, fabrication processes and materials.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the embodiments (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosed embodiments and does not pose a limitation on the scope of the disclosure unless otherwise claimed. No language in the specification should be construed as indicating any non-claimed element as essential to the practice of the embodiments.

Exemplary embodiments are described herein. Variations of those exemplary embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, the scope of the disclosure includes all modifications and equivalents of the subject matter recited herein and in the claims appended hereto as permitted by applicable law. Moreover, any combination of the above-described elements in all possible variations thereof is encompassed by the disclosure unless otherwise indicated herein or otherwise clearly contradicted by context.

1. A method of making a flexible magnetic resonance imaging (MRI) receive coil device optimized for acoustic transparency, the receive coil device having at least one receive coil and at least one capacitor, the method comprising:
a) providing a flexible substrate having a first surface and a second surface opposite the first surface;
b) forming a conductor pattern on one or both of the first and second surfaces using a conductive material, the conductor pattern including the at least one receive coil and the at least one capacitor; and
c) depositing a layer of material over at least the at least one capacitor, the layer of material having an acoustic impedance value between an acoustic impedance value of water and an acoustic impedance value of the conductive material.

2. The method of claim 1, wherein the flexible substrate comprises a dielectric plastic material selected from the group consisting of a polyimide (PI) film, a polyethylene (PE) film, a polyethylene terephthalate (PET) film, a polyethylene naphthalate (PEN) film, a polyetherimide (PEI) film, a polyphenylene sulfide (PPS) film, a polytetrafluoroethylene (PTFE) film, and a polyether ether ketone (PEEK) film.

3. The method of claim 1, further comprising coating the receive coil device with a hydrophobic material.

4. The method of claim 1, wherein forming the conductor pattern includes:
printing a first layer of the conductive material on the first surface using a printing mask having a pattern; and
printing a second layer of the conductive material on the second surface using said printing mask, wherein a portion of the first conductor pattern on the first surface overlaps with a portion of the second conductor pattern on the second surface with the flexible substrate therebetween to form the at least one capacitor.

5. The method of claim 1, The method of claim 4, wherein printing includes screen printing.

6. The method of claim 1, wherein the conductive material comprises a conductive ink.

7. The method of claim 6, wherein the conductive ink includes a metal material selected from the group consisting of gold, copper and silver.

8. The method of claim 7, wherein the metal material comprises metallic flakes.

9. The method of claim 1, wherein the conductive material includes a metal material and a polymer-based binder.

10. The method of claim 1, wherein the depositing includes depositing the layer of material over both the at least one receive coil and the at least one capacitor.

11. The method of claim 1, wherein the at least one receive coil and the at least one capacitor are substantially transparent to ultrasound frequencies.

12. The method of claim 1, wherein the layer of material includes polytetrafluoroethylene (PTFE).

13. The method of claim 1, wherein the PTFE layer has a thickness of between about 75 μm and about 500 μm.

14. The method of claim 1, wherein the conductor material includes a combination of a printed conductor material and a non-printed conductor material.

15. A method of making a flexible magnetic resonance imaging (MRI) receive coil device optimized for acoustic transparency, the receive coil device having at least one receive coil and at least one capacitor, the method comprising:
a) providing a flexible substrate having a first surface and a second surface opposite the first surface, the flexible substrate comprising a polyether ether ketone (PEEK) film;
b) forming a conductor pattern on one or both of the first and second surfaces using a conductive material, the conductor pattern including the at least one receive coil and the at least one capacitor; and
c) depositing a layer of polytetrafluoroethylene (PTFE) over at least the at least one capacitor.

16. The method of claim 15, wherein the conductive material includes a conductive ink and wherein forming the conductor pattern includes:
printing a first layer of the conductive ink on the first surface using a printing mask having a pattern; and
printing a second layer of the conductive ink on the second surface using said printing mask, wherein a portion of the first conductor pattern on the first surface overlaps with a portion of the second conductor pattern on the second surface with the flexible substrate therebetween to form the at least one capacitor.

17. The method of claim 15, wherein the conductive material includes a metal material selected from the group consisting of gold, copper and silver.

18. The method of claim 17, wherein the metal material comprises metallic flakes.

19. The method of claim 15, wherein the conductive material includes a metal material and a polymer-based binder.

20. The method of claim 15, wherein the depositing includes depositing the layer of PTFE over the at least one receive coil and the at least one capacitor.
